(19) United States
(12) Patent Application Publication
(10) Pub. No.: US 2025/0345207 A1
(43) Pub. Date: Nov. 13, 2025

(54) SLEEVES FOR OPHTHALMIC SURGICAL INSTRUMENTS

(71) Applicant: Alcon Inc., Fribourg (CH)

(72) Inventor: Nanhong Lou, Southlake, TX (US)

(21) Appl. No.: 19/180,369

(22) Filed: Apr. 16, 2025

Related U.S. Application Data
(60) Provisional application No. 63/645,216, filed on May 10, 2024.

(51) Int. Cl. A61F 9/007

(52) U.S. Cl. CPC A61F 9/00736

(57) ABSTRACT

A sleeve for an ophthalmic surgical instrument is provided. The sleeve includes a sleeve body configured to be removably disposed around the ophthalmic surgical instrument and through an incision in an eye. The sleeve body includes an inner surface and an outer surface. The inner surface defines a passage configured to receive the ophthalmic surgical instrument. The outer surface is configured to interface with surrounding tissues of the incision and reduce fluid leakage through the incision from within the eye.

### INTRODUCTION

During ophthalmic procedures, one or more incisions may be made in an eye to provide surgical instruments access to an interior portion of the eye. Once the surgical instruments are inserted into the interior portion, a user (e.g., a surgeon or an assistant) may maneuver the ophthalmic surgical instruments to cut, move, and/or remove optical tissue.

In some procedures, fluid within the eye may leak out of the eye through the incision(s) while the surgical instruments are inserted through the incision(s), thereby causing the eye's intraocular pressure (IOP) to decrease, and the potential collapse of the eye's anterior chamber (AC). To counteract the fluid loss and maintain stable IOP and AC depth, fluids may be pumped into (and/or out of) the interior portion of the eye. However, extensive fluid exchanging into and/or out of the eye may result in unwanted trauma to ocular tissue and/or other complications.

It would, therefore, be an advancement in the art to reduce the amount of fluid exchanged into and/or out of the eye during ophthalmic procedures.

### BRIEF SUMMARY

The present disclosure relates generally to sleeves for ophthalmic surgical instruments.

In certain embodiments, a sleeve for an ophthalmic surgical instrument is provided. The sleeve includes a sleeve body configured to be removably disposed around the ophthalmic surgical instrument and through an incision in an eye. The sleeve body includes an inner surface defining a passage configured to receive the ophthalmic surgical instrument and an outer surface, and an outer surface configured to interface with surrounding tissues of the incision and reduce fluid leakage through the incision from within the eye.

In certain embodiments, another sleeve for an ophthalmic surgical instrument is provided. The sleeve includes an elastomeric sleeve body configured to be removably disposed around the ophthalmic surgical instrument and through an incision in an eye. The sleeve body includes a tapered proximal end, a tapered distal end opposite the proximal end, an inner surface extending between the proximal end to the distal end, the inner surface defining a passage configured to receive the ophthalmic surgical instrument, an outer surface extending between the proximal end to the distal end, the outer surface configured to interface with surrounding tissues of the incision and reduce fluid leakage through the incision from within the eye, and a thickness between the inner surface and the outer surface, wherein the thickness is substantially uniform from the proximal end to the distal end.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1A is an isometric view of an example sleeve for an ophthalmic surgical instrument, according to certain embodiments.

FIG. 1B is an axial front-to-back view of the sleeve shown in FIG. 1A, according to certain embodiments.

FIG. 2A is an isometric view of an ophthalmic surgical instrument with the sleeve shown in FIG. 1A, according to certain embodiments.

FIG. 2B shows the ophthalmic surgical instrument of FIG. 2A inserted into an eye, according to certain embodiments.

FIG. 3A is an isometric view of another example sleeve, according to certain embodiments.

FIG. 3B is an axial front-to-back view of the sleeve shown in FIG. 3A, according to certain embodiments.

FIGS. 4A-4C are isometric views of other example sleeves, according to certain embodiments.

FIG. 5A is a cross-sectional side view of an example ophthalmic valve, according to certain embodiments.

FIG. 5B is an axial front-to-back view of the ophthalmic valve shown in FIG. 5A, according to certain embodiments.

FIG. 6A shows the ophthalmic valve of FIG. 5A inserted into an eye, according to certain embodiments.

FIG. 6B shows an ophthalmic surgical instrument inserted through the ophthalmic valve shown in FIG. 6A, according to certain embodiments.

FIG. 7 is a cross-sectional side view of another example ophthalmic valve, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a surgeon or console during an ophthalmic procedure; and the term “proximal” refers to the system, device, component, end, portion, or segment that is disposed further from the patient and/or closer to the surgeon or console during the ophthalmic procedure.

Phacoemulsification (i.e., “phaco”) is a common procedure used for the restoration of vision in people with cataracts or clouding of the lens of the eye. Current phacoemulsification techniques employ the use of a main incision, through which a phaco probe may be inserted, and a sideport incision, through which a phaco chopper may be inserted. The phaco probe is used to trench, emulsify, and aspirate lens tissues, and may further provide irrigation to maintain stability of the eye's intraocular pressure (IOP) and anterior chamber (AC). Meanwhile, the phaco chopper is used to chop and move pieces of the lens tissues and may also be used to fixate the eye during the procedure.

However, because fluids within the eye can leak from a main incision and/or a sideport incision during the use of such instruments, more fluid may need to be irrigated into the eye to maintain a stable IOP and AC. Such exchange of fluid into and out of the eye can result in unwanted and unintentional trauma to ocular tissues, which can lead to potentially permanent damage to the eye, disturbing its function and/or causing various other complications. As such, current cataract surgery techniques present a variety of limitations.

Certain embodiments described herein provide sleeves for use with surgical instruments during ophthalmic procedures. More particularly, certain embodiments provide sleeves that safely and efficiently seal incisions around surgical instruments inserted into the eye, thereby facilitating the maintenance of IOP and AC by reducing fluid leakage from within the eye through the incisions.

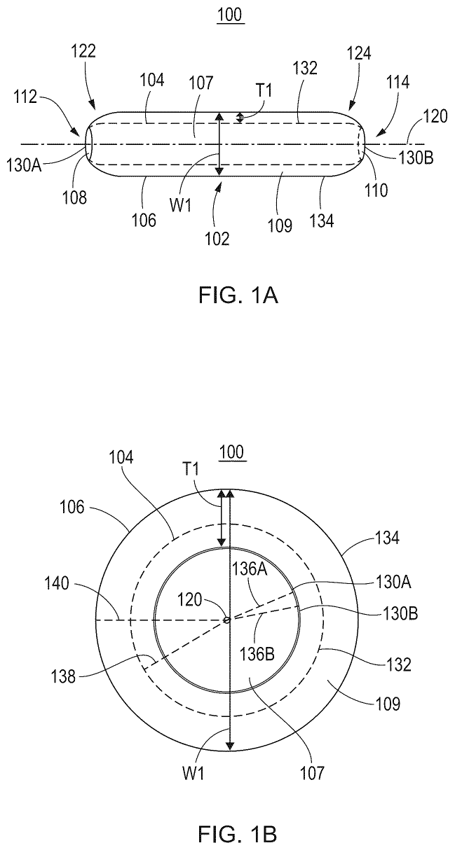

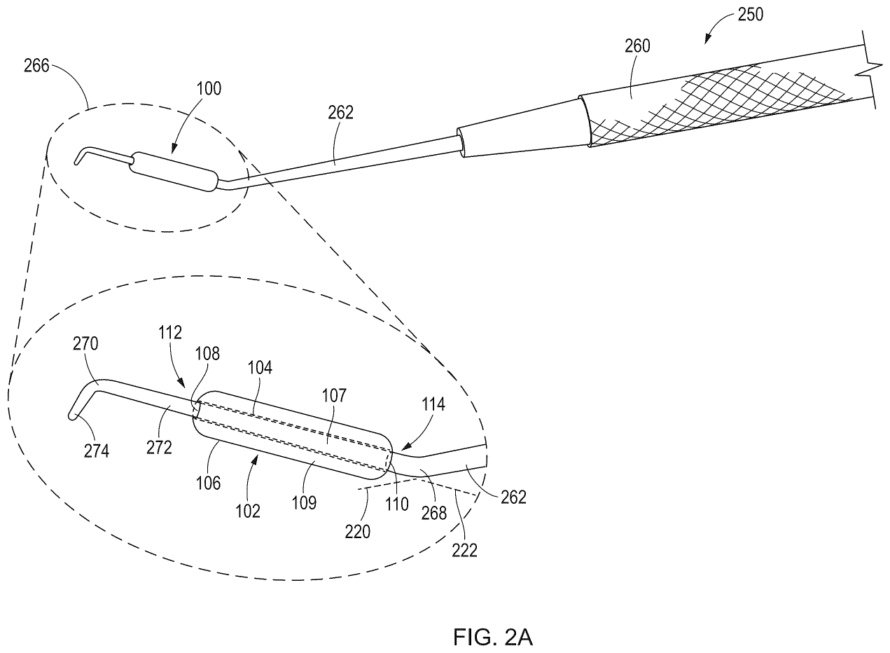

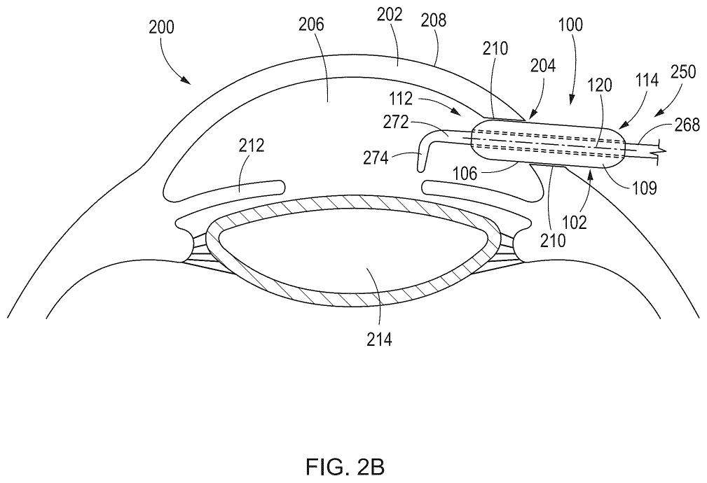

FIG. 1A is an isometric view of an example sleeve 100 for an ophthalmic surgical instrument (shown in FIG. 2A), according to certain embodiments. FIG. 1B is an axial front-to-back view of the sleeve 100 shown in FIG. 1A, according to certain embodiments. Accordingly, FIGS. 1A-1B are described together herein for clarity.

The sleeve 100 includes a sleeve body 102 with an inner surface 104, an outer surface 106, a first opening 108 at a distal end 112, and a second opening 110 at a proximal end 114. In the embodiments illustrated in FIG. 1A, an outer shape of the sleeve 100 is substantially cylindrical between the distal end 112 and the proximal end 114 but includes a first tapered portion 122 at the distal end 112, and a second tapered portion 124 at the proximal end 114. As shown, the outer surface 106 tapers (or curves) inwards towards a major longitudinal axis 120 of the sleeve 100 at the distal end 112 and the proximal end 114. The inner surface 104 is also tapered at the distal end 112 and the proximal end 114 to facilitate insertion of a surgical instrument into the sleeve 100. In some embodiments, a length of the sleeve 100 between the distal end 112 and the proximal end 114 is between 0.1 millimeters (mm) and 20 mm (e.g., between 0.5 mm and 19.5 mm, 1 mm and 19 mm, 1.5 mm and 18.5 mm, or 2 mm and 18 mm).

The inner surface 104 defines a passage 107 through the sleeve 100, the passage 107 extending the length of the sleeve 100 between the first opening 108 and the second opening 110. The passage 107 is configured to receive an ophthalmic surgical instrument (e.g., a phaco chopper or ophthalmic chopper), allowing the sleeve 100 to be removably disposed around the ophthalmic surgical instrument, as described in further detail with reference to FIG. 2A. When the ophthalmic surgical instrument with the sleeve 100 is inserted through an incision in an eye (shown in FIG. 2B), the outer surface 106 interfaces with surrounding tissues of the incision and reduces and/or prevents fluid leakage through the incision around the ophthalmic surgical instrument, as described in further detail with reference to FIG. 2B.

As shown in FIG. 1A, the inner surface 104 and the outer surface 106 define a wall 109 therebetween, the wall 109 extending circumferentially around the major longitudinal axis 120 along a length of the sleeve 100 between the first opening 108 and the second opening 110. The thickness of the wall 109 between the inner surface 104 and the outer surface 106 is substantially uniform but for the distal end 112 and the proximal end 114. In particular, the thickness of the wall 109 tapers, or is gradually reduced, at both of the ends 112, 114, as shown in FIG. 1A.

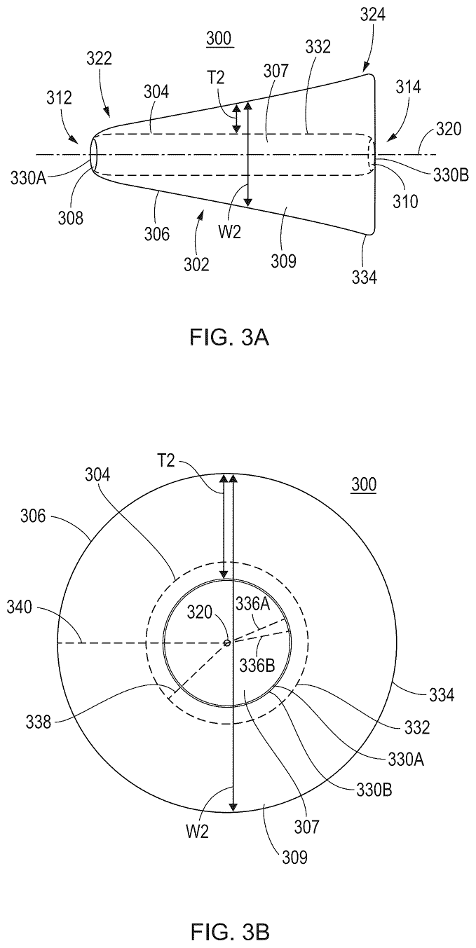

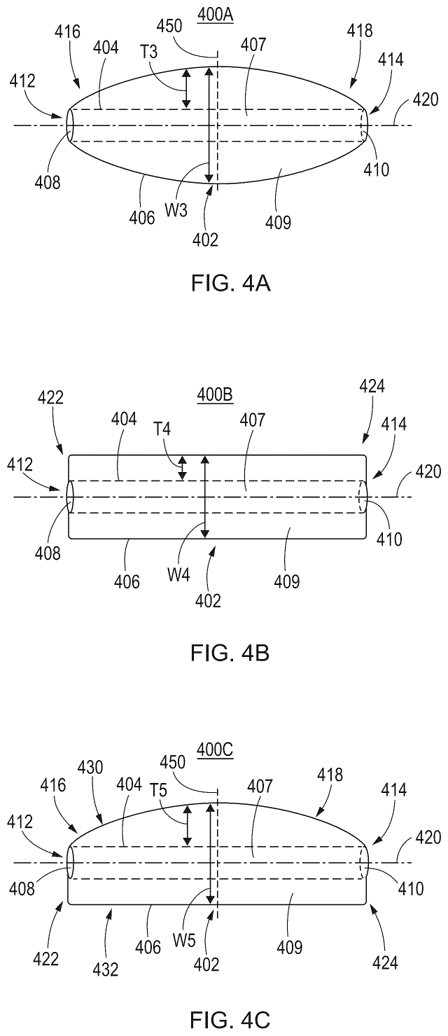

In some other embodiments, a thickness (T1) of the wall 109 between the inner surface 104 and the outer surface 106 can be substantially uniform between the distal end 112 and the proximal end 114 of the sleeve 100, as described in further detail with reference to FIG. 4B. In yet some other embodiments, the thickness may vary from the distal end 112 to the proximal end 114 of the sleeve 100, such that the thickness increases and/or decreases between the ends 112, 114. Further, although the thickness of the wall 109 tapers at both ends 112, 114, as shown in FIG. 1A, only one end (e.g., distal end 112 or proximal end 114) could be tapered in certain other embodiments. Examples of sleeves with varying thickness are described in further detail with reference to FIGS. 3A, 4A, and 4C.

In some embodiments, the thickness of the wall 109 is less than a diameter or a width of an incision (e.g., less than or equal to 2 mm). As an example, the thickness (T1) of the wall 109 may be between 0.05 mm and 1.0 mm (e.g., between 0.07 mm and 0.98 mm, 0.09 mm and 0.96 mm, 0.11 mm and 0.94 mm, or 0.13 mm and 0.92 mm). In some embodiments, the thickness of the wall 109 at tapered portions 122, 124 is less than the thickness of the wall 109 between the tapered portions 122, 124. As an example, the thickness of the wall 109 at tapered portions 122, 124 may be between 0.01 mm and 0.5 mm (e.g., between 0.03 mm and 0.48 mm, 0.05 mm and 0.46 mm, 0.07 mm and 0.44 mm, or 0.09 mm and 0.42 mm). As such, the thickness of the wall 109 gradually decreases by a change of up to 0.99 mm at the ends 112, 114 where the tapered portions 122, 124 start. In some embodiments, a width (W1) of the sleeve 100 may be between 0.15 mm and 2 mm (e.g., between 0.16 mm and 1.99 mm, 0.17 mm and 1.98 mm, 0.18 mm and 1.97 mm, or 0.19 mm and 1.96 mm).

In certain embodiments, the sleeve body 102 may be formed of an elastomeric material (e.g., silicone material), rubber, or other similar material. In such embodiments, the sleeve 100 may be flexible and configured to bend according to a shape of an ophthalmic surgical instrument (e.g., a phaco chopper) that the sleeve 100 is configured to receive. When the sleeve 100 is disposed around the ophthalmic surgical instrument (as shown in FIG. 2A), the elastomeric material's flexibility may also allow the wall 109 of the sleeve 100 to compress and decompress from any external forces acting upon the surfaces 104, 106 (e.g., gripping/compressive forces from a user or an incision in an eye). The compression and decompression of the sleeve 100 are described in further detail with reference to FIG. 2B.

The openings 108, 110 define first circumferences 130A and 130B (collectively referred to as circumference 130) having first radii 136A-136B (collectively referred to as first radius 136), respectively. Further, the inner surface 104 defines a second circumference 132 having a second radius 138, and the outer surface 106 defines a third circumference 134 having a third radius 140. In the embodiments of FIG. 1B, the radius 136A is the same size as the radius 136B. Further, the first radius 136 is smaller than the second radius 138 and the third radius 140, the second radius 138 is smaller than the third radius 140 and larger than the first radius 136, and the third radius 140 is larger than the first radius 136 and the second radius 138. As shown in FIGS. 1A-1B, the first radius 136 is smaller than the second radius 138 and the third radius 140 due to the tapered portions 122, 124.

The first radius 136 may be between, for example, 0.01 mm and 1.5 mm (e.g., between 0.02 mm and 1.4 mm, 0.03 mm and 1.3 mm, 0.04 mm and 1.2 mm, or 0.05 mm and 1.1 mm). The second radius 138 may be between, for example, 0.10 mm and 2 mm (e.g., between 0.11 mm and 1.9 mm, 0.12 mm and 1.9 mm, 0.13 mm and 1.7 mm, or 0.14 mm and 1.6 mm). The third radius 140 may be between, for example, 0.20 mm and 4 mm (e.g., between 0.3 mm and 3.9 mm, 0.4 mm and 3.8 mm, 0.5 mm and 3.7 mm, or 0.6 mm and 3.6 mm). In some embodiments, the radii 136, 138, 140 may be configured according to different incision sizes and/or surgical instruments.

Although in FIG. 1B the first circumference 130 is shown as being smaller than the second circumference 132, the first circumference 130 may expand to at least substantially match the second circumference 132 in size when the sleeve 100 is disposed around an ophthalmic surgical instrument. In other words, the elastomeric material of the sleeve 100 is configured to allow the first circumference 130 to stretch (or enlarge) such that the openings 108, 110 fit around (or over) the ophthalmic surgical instrument. Similarly, when the sleeve 100 is inserted into an incision in an eye (e.g., as shown in FIG. 2B), the elastomeric material of the sleeve 100 is configured to allow the outer surface 106 to compress in response to external forces acting on the sleeve 100 (e.g., surrounding tissues of the incision pressing against the sleeve). As such, when the outer surface 106 compresses, the third circumference 134 shrinks in size.

When the sleeve 100 is disposed around the ophthalmic surgical instrument, tensile forces of the sleeve 100's elastomeric material help create a seal between the inner surface 104 and the ophthalmic surgical instrument at least at the openings 108, 110. The tensile forces of the sleeve 100 may also help maintain a position of the sleeve 100 on the ophthalmic surgical instrument during ophthalmic procedures. An example of the sleeve 100 removably disposed around an ophthalmic surgical instrument is described in further detail with reference to FIG. 2A.

Further, the circumference 130A may be smaller than the circumference 130B, and the radius 136A of circumference 130A may be smaller than the radius 136B of circumference 130B, or vice versa. For example, when a shaft of an ophthalmic surgical instrument on which the sleeve 100 is to be disposed varies in size, such that a distal end of the shaft is narrower than a proximal end of the shaft, the circumference 130A is smaller than circumference 130B to provide a better fit and/or seal between the sleeve 100 and the ophthalmic surgical instrument.

FIG. 2A is an isometric view of an ophthalmic surgical instrument 250 with the sleeve 100 shown in FIG. 1A coupled thereto, according to certain embodiments. Although the ophthalmic surgical instrument 250 shown in FIG. 2A is a phaco chopper (sometimes referred to herein as an “ophthalmic chopper”), the ophthalmic surgical instrument 250 may be representative of other ophthalmic surgical instruments. For example, the sleeve 100 may also be implemented with an intraocular lens (IOL) manipulator.

The ophthalmic surgical instrument 250 shown in FIG. 2A includes a handle 260 with a shaft 262 having a tool portion 266. The tool portion 266 includes a first bend 268 and a second bend 270 with a substantially straight portion 272 (i.e., arm) disposed therebetween, and a tip 274 extending from the second bend 270. The straight portion 272 is angled between approximately 10° (degrees) to 80° (e.g., between 15° to 75°, 20° to 70°, or 25° to) 65° relative to a major longitudinal axis 220 of the shaft 262 and/or ophthalmic surgical instrument 250, and the tip 274 is angled approximately 90° (e.g., 90°±30°) relative to a major longitudinal axis 222 of the straight portion 272.

In the embodiments of FIG. 2A, the sleeve body 102 is configured to be removably disposed around the ophthalmic surgical instrument 250 such that the inner surface 104 interfaces with the straight portion 272 of the shaft 262 between the first bend 268 and the second bend 270. As shown in FIG. 2A, the sleeve 100 is disposed along a portion (e.g., at least 10%, 25%, 50%, or 75% or more) of a length of the straight portion 272. However, although the sleeve 100 is shown as being disposed along a portion of the straight portion 272, the sleeve 100 may also be disposed along the entire length of the straight portion 272 (e.g., between the first bend 268 and the second bend 270).

In certain embodiments, the sleeve 100 may be slid onto the ophthalmic surgical instrument 250 by pushing the tip 274 into the second opening 110 at the proximal end 114, through the passage 107, and out the first opening 108 at the distal end 112. In some embodiments, the proximal end 114 of the sleeve 100 may be configured to engage with the first bend 268 to prevent the sleeve 100 from sliding past the first bend 268 and/or further up the shaft 262 when the ophthalmic surgical instrument 250 is inserted through an incision in the eye, as described with reference to FIG. 2B.

FIG. 2B shows the ophthalmic surgical instrument 250 of FIG. 2A inserted into an eye 200, according to certain embodiments. For clarity purposes, reference numbers for the eye 200 are only shown for the cornea 202 with an outer surface 208, an interior portion 206 (e.g., an anterior chamber), an iris 212, and a lens 214.

In FIG. 2B, the ophthalmic surgical instrument 250 is introduced into the eye 200 via a clear corneal incision, such as incision 204. The incision 204 is a sideport incision configured to provide the ophthalmic surgical instrument 250 access to the interior portion 206 of the eye 200. In certain embodiments, there is another incision in addition to the incision 204, the other incision serving as a main incision through which a primary ophthalmic surgical instrument (e.g., a phacoemulsification probe or other larger ophthalmic surgical instrument) may be inserted.

As described herein, the incision 204 (e.g., the sideport incision) may feature a smaller incision (relative to the main incision) which may be used for smaller surgical tools (e.g., a phaco chopper). As an example, the width of the incision 204 is between approximately 0.5 mm and 2 mm (e.g., between 0.6 mm and 1.9 mm, 0.7 mm and 1.8 mm, or 0.8 mm and 1.7 mm).

Once the ophthalmic surgical instrument 250 is introduced into the eye 200 as shown in FIG. 2B, the outer surface 106 of the sleeve body 102 interfaces with surrounding tissues 210 of the incision 204. That is, when the ophthalmic surgical instrument 250 with the sleeve 100 is inserted through the incision 204, the surrounding tissues 210 of the incision 204 press against the outer surface 106, such that inward forces resulting from the surrounding tissues 210 compress the wall 109 inwards toward the major longitudinal axis 120 of the sleeve 100. In turn, decompression or responsive forces of the wall 109 push outwards against the surrounding tissues 210, forming a seal between the outer surface 106 of the sleeve 100 and the surrounding tissues 210 of the incision 204.

In some embodiments, the seal between the outer surface 106 and the surrounding tissues 210 helps to reduce and/or prevent fluid in the interior portion 206 of the eye 200 from leaking through the incision 204. By preventing fluid leakage through the incision 204, the amount of fluid needed to be irrigated into the eye 200 throughout the ophthalmic procedure (e.g., to maintain IOP and AC stability) can be reduced. And, with reduced fluid exchange, the likelihood of trauma to ocular tissues as caused by fluid exchange can be reduced or eliminated.

FIG. 3A is an isometric view of another example sleeve 300, according to certain embodiments. FIG. 3B is an axial front-to-back view of the sleeve 300 shown in FIG. 3A, according to certain embodiments. Accordingly, FIGS. 3A-3B are described together herein for clarity.

The sleeve 300 is another embodiment of the sleeve described with reference to FIGS. 1A-1B and may be used instead of the sleeve 100 disposed around the ophthalmic surgical instrument 250 described with reference to FIGS. 2A-2B. In certain aspects, the sleeve 300 is similar to the sleeve 100, and includes a sleeve body 302 with an inner surface 304, an outer surface 306, a first opening 308 at a distal end 312, a second opening 310 at a proximal end 314, and a passage 307 similar to the sleeve 100 as described with reference to FIGS. 1A-1B.

However, as shown in FIG. 3A, the sleeve body 302 is at least substantially conical between the distal end 312 and the proximal end 314. The conical shape of the sleeve 300 enables the sleeve 300 to function like a plug when inserted into an incision in an eye (e.g., similar to as shown in FIG. 2B). For example, when an ophthalmic surgical instrument with the sleeve 300 is inserted through the incision, the distal end 312 of the sleeve 300 may fit through the incision, but the thicker and/or wider proximal end 314 (or a portion therebetween) may press against and interface with an outer surface (e.g., outer surface 208) of the incision, like a plug or stopper. This thicker portion of the sleeve body 302 therefore helps provide a seal between the outer surface 306 and the surrounding tissues by increasing an amount of surface area contacted by the wall 309 within the incision, thereby reducing and/or preventing fluid leakage around the ophthalmic surgical instrument.

Due to the conical shape of the sleeve 300, the wall 309 of the sleeve body 302 has a thickness (T2) between the inner surface 304 and the outer surface 306 which gradually increases from the distal end 312 to the proximal end 314. In certain embodiments, the thickness at the distal end 312 may be less than a size of an incision (e.g., less than or equal to 2 mm), and the thickness at the proximal end 314 may be greater than the size of the incision (e.g., greater than 2 mm). For example, a thickness of the wall 309 at the distal end 312 may be between 0.01 mm and 0.5 mm (e.g., between 0.03 mm and 0.48 mm, 0.05 mm and 0.46 mm, 0.07 mm and 0.44 mm, or 0.09 mm and 0.42 mm), and a thickness of the wall 309 at the proximal end 314 may be between 0.50 mm and 4.0 mm (e.g., between 0.70 mm and 3.9 mm, 0.90 mm and 3.8 mm, 1.1 mm and 3.7 mm, or 1.3 mm and 3.6 mm).

In some embodiments, a width (W2) of the sleeve body 302 may increase from 0.05 mm to up to 4.0 mm from the distal end 312 to the proximal end 314. In some embodiments, a length of the sleeve 300, between the distal end 312 and the proximal end 314, is between 0.1 mm and 20 mm (e.g., between 0.5 mm and 19.5 mm, 1 mm and 19 mm, 1.5 mm and 18.5 mm, or 2 mm and 18 mm).

As shown in FIG. 3A, the sleeve body 302 also includes a tapered portion 322 at the distal end 312 and a non-tapered portion 324 at the proximal end 314. As such, the outer surface 306 may be tapered (or rounded) inwards towards a major longitudinal axis 320 of the sleeve 300 at the distal end 312, but not tapered at the proximal end 314.

The openings 308 and 310 define first circumferences 330A and 330B (collectively referred to as circumference 330) having first radii 336A-336B (collectively referred to as first radius 336), respectively. Further, the inner surface 304 defines a second circumference 332 having a second radius 338. A third circumference 334 having a third radius 340 represents the circumference of the outer surface 306 at the proximal end 314. Due to the varying thickness of the wall 309, the third circumference 334 representing the outer surface 306 may decrease to the size of the circumference 330A at the distal end 312. In other words, the circumference of the outer surface 306 decreases from the proximal end 314 to the distal end 312, such that the circumference gradually decreases in size from the third circumference 334 to the first circumference 330.

The first radius 336 may be between, for example, 0.01 mm and 1.5 mm (e.g., between 0.02 mm and 1.4 mm, 0.03 mm and 1.3 mm, 0.04 mm and 1.2 mm, or 0.05 mm and 1.1 mm). The second radius 338 may be between, for example, 0.10 mm and 2 mm (e.g., between 0.11 mm and 1.9 mm, 0.12 mm and 1.9 mm, 0.13 mm and 1.7 mm, or 0.14 mm and 1.6 mm). The third radius 340 increase in size, for example, from 0.20 mm to 4 mm (e.g., from 0.30 mm to 3.9 mm, 0.40 mm to 3.8 mm, 0.50 mm to 3.7 mm, or 0.60 mm to 3.6 mm). In some embodiments, the radii 336, 338, 340 may be configured according to different incision sizes and/or surgical instruments.

Although the first circumference 330 is shown as being smaller than the second circumference 332, the first circumference 330 may expand to match a size of the second circumference 332 when the sleeve 300 is disposed around an ophthalmic surgical instrument. In other words, the elastomeric material of the sleeve 300 allows the first circumference 330 to stretch (or enlarge) such that the openings 308, 310 fit around (or over) the ophthalmic surgical instrument. When the sleeve 300 is disposed around the ophthalmic surgical instrument, tensile forces of the sleeve 300's elastomeric material help create a seal between the inner surface 304 and the ophthalmic surgical instrument at least at the openings 308, 310. The tensile forces of the sleeve 300 may also help maintain a position of the sleeve 300 on the ophthalmic surgical instrument during ophthalmic procedures.

FIGS. 4A-4C are isometric views of other example sleeves, according to certain embodiments. The sleeves shown in FIGS. 4A, 4B, and 4C may be implemented as alternatives to the sleeve 100 shown in FIGS. 1A-1B or the sleeve 300 shown in FIGS. 3A-3B.

Turning to FIG. 4A, an isometric view of sleeve 400A for an ophthalmic surgical instrument (shown in FIG. 2A) is shown. The sleeve 400A is another embodiment of the sleeve described with reference to FIGS. 1A-1B and may be used instead of the sleeve 100 disposed around the ophthalmic surgical instrument 250 described with reference to FIGS. 2A-2B. In certain aspects, the sleeve 400A includes a sleeve body 402 with an inner surface 404, an outer surface 406, a first opening 408 at a distal end 412, a second opening 410 at a proximal end 414, and a passage 407 along a major longitudinal axis 420 of the sleeve 400A similar to the sleeve 100.

As opposed to the sleeve 100, the sleeve body 402 of the sleeve 400A is at least substantially elliptical (or oval-like) between the distal end 412 and the proximal end 414, such that the sleeve body 402 includes a tapered distal half 416 and a tapered proximal half 418. In other words, the outer surface 406 tapers inwardly toward the major longitudinal axis 420 between a midpoint axis 450 and each of the ends 412, 414. The midpoint axis 450 is positioned along the middle of the sleeve 400A, orthogonal to the major longitudinal axis 420.

To form the elliptical shape of the sleeve 400A, a wall 409 of the sleeve body 402 has a thickness (T3) between the inner surface 404 and the outer surface 406 which gradually decreases from the midpoint axis 450 to the distal end 412, and from the midpoint axis 450 to the proximal end 414. In certain embodiments, the thickness at the ends 412, 414 may be less than a size of an incision (e.g., less than or equal to 2 mm), and the thickness at the midpoint axis 450 may be greater than a size of an incision (e.g., greater than 1 mm or 2 mm). For example, the thickness of the wall 409 at the ends 412, 414 may be between 0.01 mm and 0.5 mm (e.g., between 0.03 mm and 0.48 mm, 0.05 mm and 0.46 mm, 0.07 mm and 0.44 mm, or 0.09 mm and 0.42 mm), and the thickness of the wall 409 at the midpoint axis 450 may be between 0.05 mm and 1.0 mm (e.g., between 0.07 mm and 0.98 mm, 0.09 mm and 0.96 mm, 0.11 mm and 0.94 mm, or 0.13 mm and 0.92 mm). In some embodiments, a width (W3) of the sleeve 400A may be between 0.15 mm and 2.0 mm (e.g., between 0.16 mm and 1.99 mm, 0.17 mm and 1.98 mm, 0.18 mm and 1.97 mm, or 0.19 mm and 1.96 mm).

The increasing thickness of the wall 409 from the ends 412, 414 to the midpoint axis 450 allows the sleeve 400A to function similar to a plug when inserted into an incision of an eye (e.g., similar to as shown in FIG. 2B). For example, when an ophthalmic surgical instrument with the sleeve 400A is inserted into the incision, the distal end 412 may fit through the incision, but the thicker portion of the sleeve body 402 adjacent the midpoint axis 450 may press against and interface with the surrounding tissues of the incision due to the larger circumference, like a plug or stopper. The thicker portion of the sleeve body 402 may help to provide an improved seal between the outer surface 406 and the surrounding tissues by increasing an amount of surface area contacted by the wall 409 within the incision, thereby reducing and/or preventing fluid leakage around the ophthalmic surgical instrument in the incision.

FIG. 4B is an isometric view of another sleeve 400B for an ophthalmic surgical instrument (shown in FIG. 2A), according to certain embodiments. The sleeve 400B is another embodiment of the sleeve described with reference to FIGS. 1A-1B and may be used instead of the sleeve 100 disposed around the ophthalmic surgical instrument 250 described with reference to FIGS. 2A-2B. In certain aspects, the sleeve 400B includes a sleeve body 402 with an inner surface 404, an outer surface 406, a first opening 408 at a distal end 412, a second opening 410 at a proximal end 414, and a passage 407 along a major longitudinal axis 420 of the sleeve 400B similar to the sleeve 100.

As opposed to the sleeve 100, the sleeve body 402 of the sleeve 400B is at least substantially cylindrical between the distal end 412 and the proximal end 414, but includes a flat distal end 422 and a flat proximal end 424. In other words, the wall 409 is equally round circumferentially about the major longitudinal axis 420, and each of the distal end 422 and the proximal end 424 include a flat endface. As such, a thickness (T4) of the wall 409 between the inner surface 404 and the outer surface 406 is substantially uniform from the distal end 412 to the proximal end 414. The thickness of the wall 409 may be, for example, between 0.05 mm and 1.0 mm (e.g., between 0.07 mm and 0.98 mm, 0.09 mm and 0.96 mm, 0.11 mm and 0.94 mm, or 0.13 mm and 0.92 mm). In some embodiments, a width (W4) of the sleeve 400A may be between 0.15 mm and 2.0 mm (e.g., between 0.16 mm and 1.99 mm, 0.17 mm and 1.98 mm, 0.18 mm and 1.97 mm, or 0.19 mm and 1.96 mm). Although the ends 412, 414 are described as having flat endfaces, in certain embodiments, one or both ends 412, 414 may include a taper, in addition or alternative to the flat endface.

The cylindrical shape and substantially uniform thickness of the sleeve 400B allows for an easy insertion/removal into and out of an incision of an eye. For example, when an ophthalmic surgical instrument with the sleeve 400B is inserted into the incision, the substantially uniform thickness of the wall 409 allows the sleeve 400B to more easily advance through the incision without hindrance. The sleeve 400B may also enable easier maneuvering of the ophthalmic surgical instrument during a surgical procedure, since the sleeve 400B may be less likely to cause unwanted tearing at the incision.

FIG. 4C is a cross-sectional isometric view of another sleeve 400C for an ophthalmic surgical instrument (shown in FIG. 2A), according to certain embodiments. The sleeve 400C is another embodiment of the sleeve described with reference to FIGS. 1A-1B and may be used instead of the sleeve 100 disposed around the ophthalmic surgical instrument 250 described with reference to FIGS. 2A-2B. In certain aspects, the sleeve 400C includes a sleeve body 402 with an inner surface 404, an outer surface 406, a first opening 408 at a distal end 412, a second opening 410 at a proximal end 414, and a passage 407 along a major longitudinal axis 420 of the sleeve 400C similar to the sleeve 100.

As opposed to the sleeve 100, the sleeve body 402 of the sleeve 400C is at least substantially elliptical (or oval-like) on a first side 430 along the major longitudinal axis 420, and at least substantially cylindrical on a second side 432 along the major longitudinal axis 420. In other words, the first side 430 is similar in shape to the sleeve body 402 of the sleeve 400A shown in FIG. 4A, and the second side 432 is similar in shape to the sleeve body 402 of the sleeve 400B shown in FIG. 4B. As such, a wall 409 of the sleeve body 402 at the first side 430 has a varying thickness, while the wall 409 at the second side 432 has a substantially uniform thickness.

In some embodiments, each of the first side 430 and the second side 432 may extend about 180 degrees about a circumference of the sleeve 400C (e.g., around the major longitudinal axis 420). In other embodiments, the first side 430 and the second side 432 may extend around disproportionate or unequal arcs about the circumference of the sleeve 400C.

The sleeve body 402 includes a tapered distal half 416 and a tapered proximal half 418 at the first side 430, whereas the second side 432 is non-tapered, with flat endfaces at the ends 422, 424. As such, the outer surface 406 on the first side 430 tapers (or rounds) inwards towards the major longitudinal axis 420 and towards the ends 412, 414 from a midpoint axis 450.

Due to the partially-elliptical and partially-cylindrical shape of the sleeve 400C, the wall 409 has a thickness (T5) which, at the first side 430, gradually decreases from the midpoint axis 450 to the distal end 412, and from the midpoint axis 450 to the proximal end 414, whereas the thickness of the wall 409 at the second side 432 is substantially uniform from the distal end 412, to the proximal end 414. The varying thickness at the first side 430 and the substantially uniform thickness at the second side 432 of the wall 409 allows the sleeve 400C to be inserted into incisions that may not be substantially circular. By having a varying shape and curvature along the outer surface 406, the sleeve 400C provides improved sealing for incisions which may be partially elongated or partially elliptical.

As an example, the thickness at the ends 412, 414 may be between 0.01 mm and 0.5 mm (e.g., between 0.03 mm and 0.48 mm, 0.05 mm and 0.46 mm, 0.07 mm and 0.44 mm, or 0.09 mm and 0.42 mm), and the thickness at the midpoint axis 450 may be between 0.05 mm and 1.0 mm (e.g., between 0.07 mm and 0.98 mm, 0.09 mm and 0.96 mm, 0.11 mm and 0.94 mm, or 0.13 mm and 0.92 mm). In some embodiments, a width (W5) of the sleeve 400C may be between 0.15 mm and 2.0 mm (e.g., between 0.16 mm and 1.99 mm, 0.17 mm and 1.98 mm, 0.18 mm and 1.97 mm, or 0.19 mm and 1.96 mm).

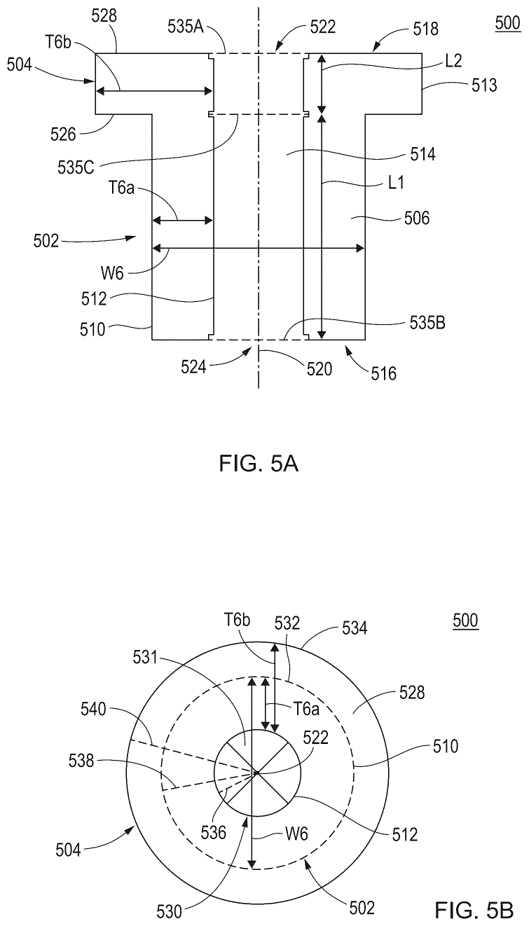

FIG. 5A is a cross-sectional side view of an example ophthalmic valve 500, according to certain embodiments. FIG. 5B is an axial front-to-back view of the ophthalmic valve 500 shown in FIG. 5A, according to certain embodiments. Accordingly, FIGS. 5A-5B are described together herein for clarity.

The ophthalmic valve 500 includes a valve shaft 502 comprising a tube 506 with an inner surface 512 and an outer surface 510, and a valve head 504 with a distal surface 526 and a proximal surface 528. The valve head 504 is a ring disposed at a proximal end 518 of the ophthalmic valve 500, and the valve shaft 502 extends from the valve head 504 to a distal end 516 of the ophthalmic valve 500. The valve shaft 502 and the valve head 504 each have at least a substantially cylindrical shape about a major longitudinal axis 520 of the ophthalmic valve 500.

The valve shaft 502 and the valve head 504 shown in FIG. 5A are integrally formed (e.g., monolithic and/or formed from a same material). One or both of the valve shaft 502 and the valve head 504 may be formed of a rigid material, such as metal or similar material. As such, the ophthalmic valve 500 may be configured to maintain its shape and facilitate easy insertion/removal of an ophthalmic surgical instrument (e.g., a phaco chopper) into and out of an eye through the ophthalmic valve 500. Further, in some embodiments, the ophthalmic valve 500 may be used in conjunction with a blade, such as a trocar blade, that can be positioned inside of the ophthalmic valve 500. The blade can create an incision in the eye, and to facilitate insertion of the ophthalmic valve 500 into the incision.

Although, in certain embodiments, the valve shaft 502 and the valve head 504 may be integrally formed, dimensions of the valve shaft 502 and the valve head 504 are described separately herein for clarity purposes. In certain other embodiments, the valve shaft 502 and the valve head 504 may be separate components rigidly or removably coupled to each other.

A thickness (T6a) of the valve shaft 502 between the outer surface 510 and the inner surface 512 (i.e., the tube 506) is substantially uniform from the distal end 516 to the distal surface 526. Further, the thickness of the tube 506 at the valve shaft 502 may be less than a diameter or a width of an incision (e.g., less than or equal to 2 mm). As such, the thickness (T6) of the tube 506 at the valve shaft 502 may be between 0.05 mm and 1 mm (e.g., between 0.07 mm and 0.98 mm, 0.09 mm and 0.96 mm, 0.11 mm and 0.94 mm, or 0.13 mm and 0.92 mm). In some embodiments, a width (W6) of the valve shaft 502 may be between 0.15 mm and 2 mm (e.g., between 0.16 mm and 1.99 mm, 0.17 mm and 1.98 mm, 0.18 mm and 1.97 mm, or 0.19 mm and 1.96 mm). In some embodiments, a length (L1) of the valve shaft 502 along the major longitudinal axis 520 may be between 0.1 mm and 15 mm (e.g., between 0.5 mm and 14 mm, 1 mm and 13 mm, 1.5 mm and 12 mm, or 2 mm and 11 mm). In some embodiments, the length may be configured so that the ophthalmic valve 500 does not contact the iris (e.g., iris 612) when inserted into the eye.

A thickness (T6b) of the tube 506 at the valve head 504 between the inner surface 512 and an outer ring surface 513 is at least substantially uniform. As an example, a thickness of the tube 506 at the valve head 504 is greater than the diameter or the width of the incision (e.g., more than 1 mm). In certain embodiments, the thickness of the tube 506 at the valve head 504 may be between 0.5 mm and 4 mm (e.g., between 0.6 mm and 3.9 mm, 0.7 mm and 3.8 mm, 0.8 mm and 3.7 mm, or 0.9 mm and 3.6 mm). In some embodiments, a length (L2) of the valve head 504 along the major longitudinal axis 520 may be between 0.1 mm and 5 mm (e.g., between 0.5 mm and 4.5 mm, 1 mm and 4.0 mm, or 1.5 mm and 3.5 mm).

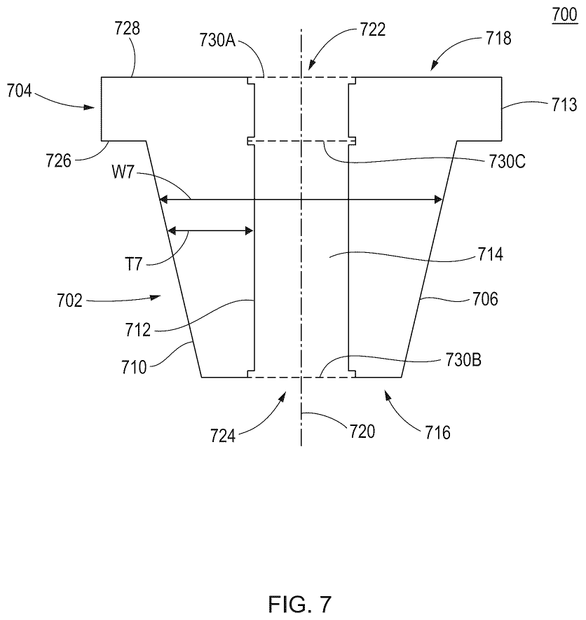

Although the thickness of the tube 506 at the valve shaft 502 is shown as substantially uniform in FIG. 5A, in some embodiments, the thickness may vary. For example, the thickness of the tube 506 at the valve shaft 502 may increase and/or decrease between the distal end 516 and the proximal end 518. An example of an ophthalmic valve with varying thickness is described in further detail with reference to FIG. 7.

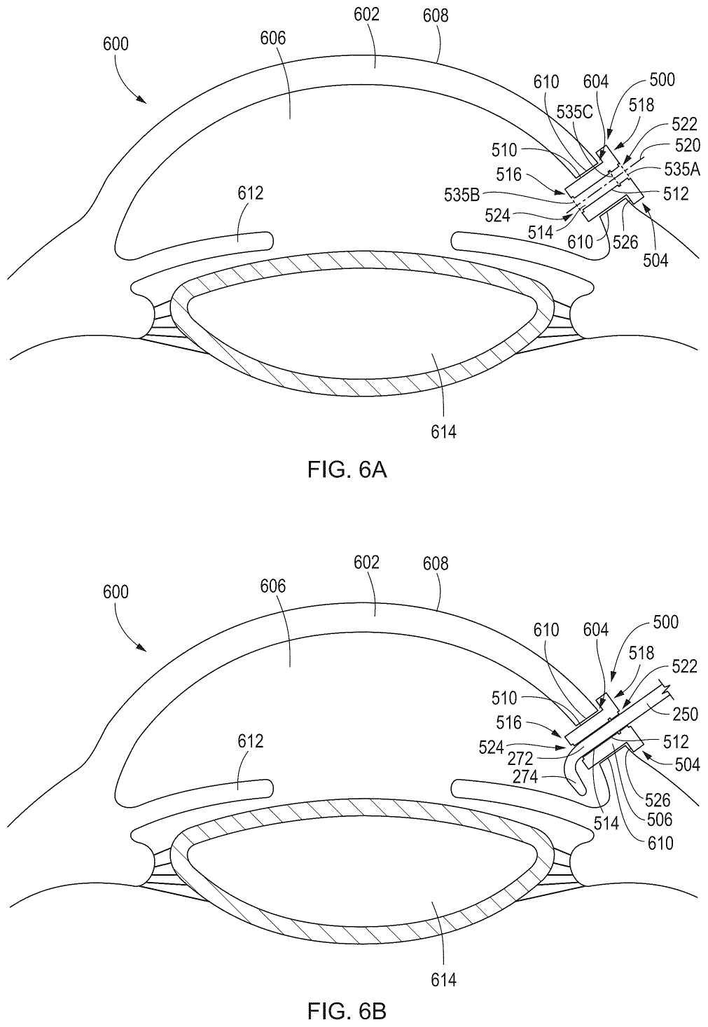

Returning to FIG. 5A, the inner surface 512 defines a passage 514 through the ophthalmic valve 500, the passage 514 extending from the distal end 516 to the proximal end 518. In certain embodiments, the passage 514 is configured to receive an ophthalmic surgical instrument (e.g., a phaco chopper), and provide the ophthalmic surgical instrument access to an interior portion of an eye, as described in further detail with reference to FIGS. 6A-6B. Thus, the ophthalmic valve 500 comprises a first passage entry 522 at the proximal end 518, and a second passage entry 524 at the distal end 516. As an example, the first passage entry 522 may be an insertion point through which the ophthalmic surgical instrument is inserted through from outside the eye, and the second passage entry 524 may be an exit point through which the ophthalmic surgical instrument is inserted through to access the interior portion of the eye.

The ophthalmic valve 500 may optionally include one or more gates 535A, 535B, 535C within the passage 514. A first gate 535A is disposed at the first passage entry 522, a second gate 535B is disposed at the second passage entry 524, and a third gate 535C is disposed in the passage 514 between the first gate 535A and the second gate 535B. In certain embodiments, the gates 535 may be formed of an elastomeric material (e.g., silicone material), rubber, or other similar material. In the embodiments of FIG. 5A, the gates 535A, 535B, 535C comprise a set of flaps 531. However, in certain other embodiments, each of the gates 535A, 535B, 535C may comprise a slit in a membrane. The gates 535A, 535B, 535C may be configured to prevent fluid in the interior portion of the eye from leaking through the passage 514, as described in further detail with reference to FIG. 6B.

Although the ophthalmic valve 500 is shown as including three gates 535A, 535B, 535C in FIG. 5A, the ophthalmic valve 500 may include more or less than three gates. Additionally, the one or more gates may be disposed at different locations within the passage 514.

Turning to FIG. 5B, the inner surface 512 and the passage entries 522, 524 define a first circumference 530 having a first radius 536, the outer surface 510 of the valve shaft 502 defines a second circumference 532 having a second radius 538, and the outer ring surface 513 of the valve head 504 defines a third circumference 534 having a third radius 540. The first radius 536 is smaller than the second radius 538 and the third radius 540, the second radius 538 is smaller than the third radius 540 and larger than the first radius 536, and the third radius 540 is larger than the first radius 536 and the second radius 538.

The first radius 536 may be between, for example, 0.1 mm and 0.3 mm (e.g., between 0.11 mm and 0.29 mm, 0.12 mm and 0.28 mm, 0.13 mm and 0.27 mm, or 0.14 mm and 0.26 mm). The second radius 538 may be between, for example, 0.2 mm and 0.5 mm (e.g., between 0.21 mm and 0.49 mm, 0.22 mm and 0.48 mm, or 0.23 mm and 0.47 mm). The third radius 540 may be between, for example, 0.3 mm and 1.0 mm (e.g., between 0.4 mm and 0.9 mm, 0.5 mm and 0.8 mm, or 0.6 mm and 0.7 mm). In some embodiments, the radii 136, 138, 140 may be configured according to different incision sizes and/or surgical instruments.

FIG. 6A shows the ophthalmic valve 500 of FIG. 5A inserted into an eye 600, according to certain embodiments. Similar to the eye 200 shown in FIG. 2B, the eye 600 is shown as including a cornea 602 with an outer surface 608, an interior portion 606 (e.g., an anterior chamber), an iris 612, a lens 614, and other eye tissues. The eye 600 also features an incision 604 which may be made prior to insertion of the ophthalmic valve 500.

In FIG. 6A, the ophthalmic valve 500 is introduced into the eye 600 via the incision 604 in the cornea 602. The incision 604 is a sideport incision configured to provide an ophthalmic surgical instrument (e.g., a phaco chopper) access to the interior portion 606 of the eye 600. As an example, the width of the incision 604 is between approximately 0.5 mm and 2 mm (e.g., between 0.6 mm and 1.9 mm, 0.7 mm and 1.8 mm, or 0.8 mm and 1.7 mm).

Once the ophthalmic valve 500 is introduced into the eye 600 as shown in FIG. 6A, the outer surface 510 of the valve shaft 502 interfaces with surrounding tissues 610 of the incision 604, and the distal surface 526 of the valve head 504 interfaces with the outer surface 608 of the cornea 602. The surrounding tissues 610 of the incision 604 then press against the outer surface 510, thereby forming a seal between the outer surface 510 of the ophthalmic valve 500 and the surrounding tissues 610 of the incision 604. Further, the valve head 504 may prevent the ophthalmic valve 500 from slipping through the incision 604 and/or from further intruding into the interior portion 606 of the eye 600.

The seal formed between the outer surface 510 and the surrounding tissues 610 helps reduce and/or prevent fluid in the interior portion 606 of the eye 600 from leaking around the ophthalmic valve 500 through the incision 604. Additionally, the one or more gates 535A, 535B, 535C prevent fluid within the interior portion 606 of the eye 600 from leaking through the passage 514 around an inserted ophthalmic surgical instrument. By preventing fluid from leaking through the incision 604 and/or through the ophthalmic valve 500, IOP within the eye 600 can be better maintained, and the amount of fluid exchanged throughout the ophthalmic procedure to stabilize IOP and AC can be reduced. And, with reduced fluid exchange, the likelihood of trauma to ocular tissues as caused by fluid exchange can be reduced or eliminated.

FIG. 6B shows the ophthalmic surgical instrument 250 inserted through the ophthalmic valve 500 shown in FIG. 6A, according to certain embodiments. As described above, the ophthalmic surgical instrument 250 may be, for example, a phaco chopper.

After the ophthalmic valve 500 has been positioned in the cornea 602, the ophthalmic surgical instrument 250 may be introduced and/or removed from the eye 600 via the passage 514 of the ophthalmic valve 500. That is, the ophthalmic valve 500 is configured to provide the ophthalmic surgical instrument 250 access to the interior portion 606 of the eye 600. And while the ophthalmic surgical instrument 250 and ophthalmic valve 500 are disposed in the eye 600, the ophthalmic valve 500 prevents fluid from leaking out between the surrounding tissues 610 and the outer surface 510, and through the passage 514 as described with reference to FIG. 6A.

Further, once the tip 274 of the ophthalmic surgical instrument 250 is positioned in the interior portion 606 of the eye 600, the ophthalmic surgical instrument 250 may be manipulated by a user (e.g., a surgeon) so as to move the tip 274 around the interior portion 606 of the eye 600. While the ophthalmic surgical instrument 250 is moved around within the eye 600, the gates 535 reduce fluid leakage through the passage 514.

FIG. 7 is a cross-sectional side view of another ophthalmic valve 700, according to certain embodiments. The ophthalmic valve 700 is another embodiment of the ophthalmic valve described with reference to FIGS. 5A-5B and may be used instead of the ophthalmic valve 500.

In certain aspects, the ophthalmic valve 700 includes a valve shaft 702 formed by a tube 706 with an inner surface 712 and an outer surface 710, a valve head 704 with a distal surface 726, a proximal surface 728, and outer ring surface 713, a passage 714 with a first passage entry 722 at a proximal end 718 of the ophthalmic valve 700, a second passage entry 724 at a distal end 716 of the ophthalmic valve 700, and three optional gates 730A, 730B, and 730C disposed within the passage 714 along a major longitudinal axis 720, similar to the ophthalmic valve 500 described with reference to FIGS. 5A-5B.

However, as opposed to the ophthalmic valve 500, the valve shaft 702 of the ophthalmic valve 700 is at least substantially conical between the distal end 716 and the valve head 704. As such, the outer surface 710 tapers (or slopes) inwards towards a major longitudinal axis 320 of the ophthalmic valve 700 between the valve head 704 and the distal end 716. Due to the conical shape of the ophthalmic valve 700, the valve shaft 702 has a thickness (T7) between the outer surface 710 and the inner surface 712 which gradually increases from the distal end 716 towards the proximal end 718.

In certain embodiments, the thickness at the distal end 716 may be less than a size of an incision (e.g., less than or equal to 2 mm), and the thickness towards the proximal end 718 may be greater than the size of the incision (e.g., greater than 2 mm). For example, a thickness at the distal end 716 may be between 0.1 mm and 2 mm (e.g., between 0.12 mm and 1.98 mm, 0.13 mm and 1.97 mm, or 0.14 mm and 1.96 mm), and a thickness at the proximal end 718 may be between 2.1 mm and 5 mm (e.g., between 2.2 mm and 4.9 mm, 2.3 mm and 4.8 mm, 2.4 mm and 4.7 mm, or 2.4 mm and 4.6 mm). As such, a width (W7) of the valve shaft may increase from 0.1 mm to up to 5 mm from the distal end 716 to the proximal end 718.

The increasing thickness of the valve shaft 702 configures the ophthalmic valve 700 to function similar to a plug when inserted in an incision of an eye (e.g., similar to as shown in FIGS. 6A-6B). For example, when the ophthalmic valve 700 is inserted in the incision, the distal end 716 fits through the incision, but a thicker portion of the valve shaft 702 towards the proximal end 718 presses against an outer surface (e.g., outer surface 608) of the incision due to the larger circumference. The thicker portion of the valve shaft 702 which interfaces with surrounding tissues (e.g., surrounding tissues 610) of the incision helps provide an improved seal between the outer surface 710 and the surrounding tissues by increasing an amount of surface area contacted by the valve shaft 702 within the incision. Thus, the varying thickness of the valve shaft 702 provides an improved seal which further helps reduce and/or prevent fluid leakage through the incision from within the eye.

As used herein, the terms “substantially” and “approximately” provide an industry-accepted tolerance to its corresponding term. This tolerance may range from, for example, less than one percent to twenty percent and may include, for example, component sizes, volumes, etc. As used herein, the term “coupled” may include direct coupling and indirect coupling via another component or element.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

### Example Embodiments

Embodiment 1: An ophthalmic valve, the ophthalmic valve comprising: a valve shaft configured to be removably disposed through an incision in an eye, the valve shaft comprising: an inner surface defining a passage configured to: receive an ophthalmic surgical instrument; and provide the ophthalmic surgical instrument access to an interior portion of the eye; and an outer surface configured to interface with surrounding tissues of the incision and reduce fluid leakage through the incision from within the eye.

Embodiment 2: The ophthalmic valve of Embodiment 1, wherein the ophthalmic valve comprises one or more gates disposed within the passage.

Embodiment 3: The ophthalmic valve of Embodiment 2, wherein the one or more gates comprise a set of flaps or a slit in a membrane.

Embodiment 4: The ophthalmic valve of Embodiment 1, wherein a thickness of the valve shaft between the inner surface and the outer surface is between 0.05 millimeters (mm) and 1 millimeters.

Embodiment 5: The ophthalmic valve of Embodiment 4, wherein the thickness is substantially uniform from a distal end of the ophthalmic valve to a proximal end of the ophthalmic valve.

Embodiment 6: The ophthalmic valve of Embodiment 4, wherein the thickness varies from a distal end of the ophthalmic valve to a proximal end of the ophthalmic valve.

Embodiment 7: The ophthalmic valve of Embodiment 1, wherein a length of the ophthalmic valve between a proximal end of the ophthalmic valve and a distal end of the ophthalmic valve is between 0.1 millimeters (mm) and 20 millimeters.

Embodiment 8: The ophthalmic valve of Embodiment 1, wherein a shaft of an ophthalmic chopper is configured to be removably disposed through the ophthalmic valve.

Embodiment 9: The ophthalmic valve of Embodiment 1, wherein the ophthalmic valve is formed of a metal material.

Embodiment 10: An ophthalmic valve, the ophthalmic valve comprising: a valve shaft configured to be removably disposed through an incision in an eye, the valve shaft comprising: a proximal end; a distal end opposite the proximal end; an inner surface defining a passage configured to: receive an ophthalmic surgical instrument; and provide the ophthalmic surgical instrument access to an interior portion of the eye; an outer surface configured to interface with surrounding tissues of the incision and reduce fluid leakage through the incision from within the eye; and a thickness between the inner surface and the outer surface, wherein the thickness is substantially uniform from the proximal end to the distal end.

Embodiment 11: An ophthalmic valve, the ophthalmic valve comprising: a valve shaft configured to be removably disposed through an incision in an eye, the valve shaft comprising: a proximal end; a distal end opposite the proximal end; an inner surface defining a passage configured to: receive an ophthalmic surgical instrument; and provide the ophthalmic surgical instrument access to an interior portion of the eye; an outer surface configured to interface with surrounding tissues of the incision and reduce fluid leakage through the incision from within the eye; and a thickness between the inner surface and the outer surface, wherein the thickness increases from the proximal end to the distal end.

What is claimed is:

1. A sleeve for an ophthalmic surgical instrument, the sleeve comprising:
a sleeve body configured to be removably disposed around the ophthalmic surgical instrument and through an incision in an eye, the sleeve body comprising:
an inner surface defining a passage configured to receive the ophthalmic surgical instrument; and
an outer surface configured to interface with surrounding tissues of the incision and reduce fluid leakage through the incision from within the eye.

2. The sleeve of claim 1, wherein:
the sleeve is comprised of an elastomeric material,
the passage comprises openings defining a first circumference,
the inner surface defines a second circumference that is larger than the first circumference,
the openings are configured to stretch to fit around the ophthalmic surgical instrument, and
when the openings stretch, the first circumference expands to at least substantially match the second circumference in size.

3. The sleeve of claim 2, wherein when the openings stretch, they are configured to:
create a seal between the inner surface of the sleeve and the ophthalmic surgical instrument at least at the openings of the passage; and
maintain a position of the sleeve on the ophthalmic surgical instrument.

4. The sleeve of claim 2, wherein:
the outer surface defines a third circumference that is larger than the first circumference and the second circumference,
the outer surface is configured to compress, and
when the outer surface compresses, the third circumference shrinks in size.

5. The sleeve of claim 1, wherein a thickness of the sleeve between the inner surface and the outer surface is between 0.05 millimeters (mm) and 1 mm.

6. The sleeve of claim 5, wherein the thickness is substantially uniform from a distal end of the sleeve to a proximal end of the sleeve.

7. The sleeve of claim 5, wherein the thickness varies from a distal end of the sleeve to a proximal end of the sleeve.

8. The sleeve of claim 7, wherein the thickness tapers from the proximal end of the sleeve to the distal end of the sleeve.

9. The sleeve of claim 1, wherein a length of the sleeve between a proximal end of the sleeve and a distal end of the sleeve is between 0.1 millimeters (mm) and 20 mm.

10. The sleeve of claim 1, wherein the sleeve is configured to be removably disposed around a shaft of an ophthalmic chopper.

11. The sleeve of claim 1, wherein:
a distal end and a proximal end of the sleeve body are tapered; and
the sleeve body is substantially cylindrical between the distal end and the proximal end.

12. A sleeve for an ophthalmic surgical instrument, the sleeve comprising:
an elastomeric sleeve body configured to be removably disposed around the ophthalmic surgical instrument and through an incision in an eye, the elastomeric sleeve body comprising, comprising:
a tapered proximal end;
a tapered distal end opposite the tapered proximal end;
an inner surface extending between the tapered proximal end to the tapered distal end, the inner surface defining a passage configured to receive the ophthalmic surgical instrument;
an outer surface extending between the tapered proximal end to the tapered distal end, the outer surface configured to interface with surrounding tissues of the incision and reduce fluid leakage through the incision from within the eye; and
a thickness between the inner surface and the outer surface, wherein the thickness is substantially uniform from the tapered proximal end to the tapered distal end.

13. The sleeve of claim 12, wherein:
the sleeve is comprised of an elastomeric material,
the passage comprises openings defining a first circumference,
the inner surface defines a second circumference that is larger than the first circumference,
the openings are configured to stretch to fit around the ophthalmic surgical instrument, and
when the openings stretch, the first circumference expands to at least substantially match the second circumference in size.

14. The sleeve of claim 13, wherein when the openings stretch, they are configured to:
create a seal between the inner surface of the sleeve and the ophthalmic surgical instrument at least at the openings of the passage; and
maintain a position of the sleeve on the ophthalmic surgical instrument.

15. The sleeve of claim 12, wherein the sleeve is configured to be removably disposed around a shaft of an ophthalmic chopper.
